(19) United States
(12) Patent Application Publication
(10) Pub. No.: US 2025/0152641 A1
(43) Pub. Date: May 15, 2025

(54) LACTOBACILLUS PARACASEI NB23 STRAIN AND METHOD OF USING THE SAME TO INCREASE MUSCLE MASS AND TO PREVENT/TREAT METABOLIC SYNDROME

(71) Applicant: New Bellus Enterprises Co., Ltd., Tainan City (TW)

(72) Inventors: Hsueh-Fang Wang, Tainan City (TW); Ping-Jui Lin, Tainan City (TW); Ting-Chun Lin, Tainan City (TW); Chyou-Wei Wei, Tainan City (TW); Fang-Chung Kuo, Tainan City (TW); Wang-Sheng Ko, Tainan City (TW)

(21) Appl. No.: 18/922,867

(22) Filed: Oct. 22, 2024

(30) Foreign Application Priority Data
Nov. 9, 2023 (TW) 112143308

(51) Int. Cl. A61K 35/747; A61P 3/00; A61P 9/00; A61P 21/00

(52) U.S. Cl. CPC A61K 35/747; A61P 3/00; A61P 9/00; A61P 21/00

(57) ABSTRACT

Disclosed herein are the Lactobacillus paracasei NB23 strain and a method of using the same to increase muscle mass and to prevent/treat metabolic syndrome. Administering an effective amount of the Lactobacillus paracasei NB23 strain or a metabolite thereof to an individual can effectively produce the effect of treating or preventing diseases associated with muscle loss or insufficient muscle mass, metabolic syndrome, and diseases related thereto.

### BACKGROUND OF THE INVENTION

### 1. Technical Field

The present invention relates to a novel probiotic strain and a use thereof. More particularly, the invention relates to the Lactobacillus paracasei NB23 strain and a method of using the same to increase muscle mass and to prevent/treat metabolic syndrome.

### 2. Description of Related Art

Muscles constitute about 40%-50% of the weight of the human body, and maintenance of the metabolism and mobility of the human body is closely related to muscle mass and muscle strength. Studies have shown that muscle mass decreases on a yearly basis after the age of 30, that the rate of muscle loss increases with age, and that the muscle mass at the age of 40-50 is about 10%-20% lower than that at the age of 30. Apart from the age factor, muscle loss is associated with one's exercise habits and eating habits. A decrease in muscle mass affects not only body shape, but also an individual's mobility and physical capacity, and may cause pain in the lower back, difficulty in lifting heavy objects, slowness in motion, poor memory, and so on.

Clinically, there is currently no drug for treating sarcopenia caused by a decrease in muscle mass, so a patient with sarcopenia can only be advised to change their eating and exercise habits, i.e., to consume a larger amount of protein and receive resistance training for a longer time than before, in order to improve the symptoms of sarcopenia. Most patients, however, find it difficult to form and keep new exercise and eating habits, and this has made sarcopenia a hard-to-treat disease. It is therefore important to develop a composition that can help prevent or improve insufficient muscle mass or related diseases.

### BRIEF SUMMARY OF THE INVENTION

The primary objective of the present invention is to provide the Lactobacillus paracasei NB23 strain and a method of using the same to increase muscle mass and to prevent/treat metabolic syndrome. The Lactobacillus paracasei NB23 strain disclosed herein is a probiotic that is safe to the human body and that has the physiological activity to promote the synthesis and increase of muscle proteins, to enhance the proliferation and hypertrophy of muscle cells, to resist inflammation and oxidation, and so on. Therefore, the Lactobacillus paracasei NB23 strain has the potential of being developed into lactic acid bacterium-related products.

To achieve the foregoing objective, the present invention discloses the Lactobacillus paracasei NB23 strain and a method of using the same to increase muscle mass and to prevent/treat metabolic syndrome. The Lactobacillus paracasei NB23 strain was deposited at the Food Industry Research and Development Institute in Hsinchu City, Taiwan under deposit number BCRC911201 on Oct. 4, 2023. The Lactobacillus paracasei NB23 strain was also deposited at Deutsche Sammlung von Mikroorganismen und Zellkulturen GmbH (DSMZ) under deposit number DSM 35133 on Sep. 5, 2024.

One embodiment of the present invention discloses a composition that includes an effective amount of the Lactobacillus paracasei NB23 strain or a metabolite thereof, wherein the composition may be, for example, a food, a nutritional supplement, or a pharmaceutical composition.

For instance, when prepared as a food, the composition disclosed herein may include an effective amount of the Lactobacillus paracasei NB23 strain and an ingredient that is acceptable in food; or when prepared as a pharmaceutical composition, the composition disclosed herein may include an effective amount of the Lactobacillus paracasei NB23 strain and a pharmaceutically acceptable ingredient.

In another embodiment of the present invention, the Lactobacillus paracasei NB23 strain has the activity to inhibit nuclear factor kappa-light-chain-enhancer of activated B cells (NF-κB) and forkhead box protein-O (Fox-O) and can therefore prevent the degradation of muscle proteins or the apoptosis of muscle cells. In consequence, a composition containing the Lactobacillus paracasei NB23 strain or a metabolite thereof can be used to treat and/or prevent sarcopenia or symptoms related thereto such as muscle atrophy, muscle weakness, reduced mobility, and reduced grip strength.

In another embodiment of the present invention, the Lactobacillus paracasei NB23 strain can activate insulin-like growth factor 1 (IGF-1) and insulin, thereby activating the phosphoinositide 3-kinase (PI3K)/protein kinase B (Akt)/mammalian target of rapamycin (mTOR) pathway in order to promote protein synthesis in muscle cells. Therefore, administering a composition containing the Lactobacillus paracasei NB23 strain or a metabolite thereof to an individual can enhance the rate of muscle synthesis and thereby increase the individual's muscle mass, producing the effect of gaining muscles and improving diseases related to insufficient muscle mass.

Another embodiment of the present invention provides a method of using the Lactobacillus paracasei NB23 strain to treat or improve metabolic syndrome. More specifically, the Lactobacillus paracasei NB23 strain can reduce blood pressure, triglycerides, and fasting blood glucose level as well as increase high-density lipoprotein cholesterol. Therefore, administering a composition containing the Lactobacillus paracasei NB23 strain or a metabolite thereof to an individual can produce the effect of preventing or treating metabolic syndrome or diseases related thereto.

Still another embodiment of the present invention discloses a method of using the Lactobacillus paracasei NB23 strain to treat or improve a cardiovascular disease. As the Lactobacillus paracasei NB23 strain has the activity to resist inflammation, lower the values of cell damage indicators, and regulate cardiovascular sclerosis indicators, administering a composition containing the Lactobacillus paracasei NB23 strain or a metabolite thereof to an individual can produce the effect of preventing or treating a cardiovascular disease.

More specifically, the cardiovascular disease is caused by vascular sclerosis.

Indicators of cardiovascular sclerosis include low-density lipoprotein cholesterol (LDL-C), high-density lipoprotein cholesterol (HDL-C), the ratio of total cholesterol to low-density lipoprotein cholesterol (TC/LDL-C), the ratio of low-density lipoprotein cholesterol to high-density lipoprotein cholesterol (LDL-C/HDL-C), and vitamin D, among others.

Cell damage indicators include C-reactive protein (CRP), lactate dehydrogenase (LDH), tumor necrosis factor (TNFα), and interleukin 6 (IL-6).

### BRIEF DESCRIPTION OF THE SEVERAL VIEWS OF THE DRAWINGS

FIG. 1 shows the analysis results of the expression of PI3K protein in muscle cells that were co-cultured separately with different lactic acid bacterium strains.

FIG. 2 shows the analysis results of the expression of mTOR protein in muscle cells that were co-cultured separately with different lactic acid bacterium strains.

FIG. 3 shows the analysis results of the expression of Akt protein in muscle cells that were co-cultured separately with different lactic acid bacterium strains.

### DETAILED DESCRIPTION OF THE INVENTION

The present invention discloses the Lactobacillus paracasei NB23 strain and a method of using the same to increase muscle mass and to prevent/treat metabolic syndrome. More specifically, the Lactobacillus paracasei NB23 strain disclosed herein has the activity to activate IGF-1 and hence the PI3K/Akt/mTOR signaling pathway; to lower the values of such inflammation and cell damage indicators as CRP, LDH, TNFα, and IL-6; to lower the values of such cardiovascular sclerosis indicators/factors as LDL-C, TC/HDL-C, and LDL-C/HDL-C; to improve such cardiovascular disease indicators as HDL-C and vitamin D; and to regulate metabolic syndrome indicators, e.g., to lower the systolic pressure and fasting blood glucose level, to reduce triglycerides, and to increase HDL-C. Therefore, administering an effective amount of the Lactobacillus paracasei NB23 strain or a metabolite thereof to an individual can effectively produce the effect of treating or preventing diseases associated with muscle loss or insufficient muscle mass, metabolic syndrome, and diseases related thereto.

The Lactobacillus paracasei NB23 strain disclosed herein was deposited at the Food Industry Research and Development Institute, Hsinchu City, Taiwan under deposit number BCRC911201 on Oct. 4, 2023. The Lactobacillus paracasei NB23 strain was also deposited at. Deutsche Sammlung von Mikroorganismen und Zellkulturen GmbH (DSMZ) under deposit number DSM 35133 on Sep. 5, 2024.

The Lactobacillus paracasei NB23 strain disclosed herein was separated from plant-based pickles, was a Gram-positive bacterium, and was cultured in a Lactobacilli MRS (De Man-Rogosa-Sharpe agar) broth in an anaerobic environment, with the growing temperature being 37° C.

As used herein, the term “effective amount” refers to an amount in which a probiotic or a metabolite thereof is administered and that can alleviate or improve one or more symptoms of the disease to be treated, reduce the discomfort of one or more symptoms of the disease, or slow down the progression of the disease.

As used herein, the term “composition” refers to a substance that contains the Lactobacillus paracasei NB23 strain disclosed herein or a metabolite thereof as a main active ingredient, that can be prepared in different dosage forms depending on such factors as the target users and the method of use, and that may be added with an excipient, a vehicle, and/or other ingredients that are pharmaceutically acceptable or are acceptable in the food industry. For example, the composition may be a pharmaceutical, a nutritional supplement, a food, or a preparation having a therapeutic effect.

As used herein, the term “metabolite” refers to a byproduct of the culturing process of the Lactobacillus paracasei NB23 strain disclosed herein, wherein the byproduct may exist in the culture medium.

As used herein, the term “administer” or “administering” refers to the act or method of providing the Lactobacillus paracasei NB23 strain disclosed herein, a metabolite thereof, or a composition containing the strain or the metabolite to an individual. For example, the administration may be carried out orally or by transplantation.

The technical features of the present invention and their effects are detailed below with reference to some examples and the accompanying drawings.

The cells used in the following examples, such as C2C12 myoblasts, are biological materials easily accessible to a person of ordinary skill in the art and therefore need not be deposited at a depository for biological materials.

The contents and procedures of the human subject research in the following examples were all in compliance with the relevant ethical guidelines.

### EXAMPLE 1: MUSCLE CELL CULTIVATION

C2C12 myoblasts were placed in a culture medium and were cultured in a 5%-carbon dioxide environment at 37° C. During the culturing process, a liquid growth medium necessary for the growth of the muscle cells was supplied in a timely manner. The culture medium used was a Dulbecco's modified Eagle's medium (DMEM) containing 5% fetal bovine serum. The liquid growth medium contained 1% 4 mM L-glutamine, 1.5 g/L sodium bicarbonate, 4.5 g/L glucose, 100 μg/mL streptomycin, and 10 unit/mL penicillin and had a pH value in the range from 7.1 to 7.4.

### EXAMPLE 2: ANTI-OXIDATION TEST

The lactic acid bacteria in Table 1 were obtained and cultured. Each cultured lactic acid bacterium was disrupted together with the supernatant collected from the corresponding culture and was subsequently diluted at different dilution ratios, i.e., diluted 8 times, 16 times, 50 times, and 200 times, in order to produce differently diluted samples of each lactic acid bacterium. The C2C12 muscle cells cultured in example 1 were then treated separately with the lactic acid bacterium samples, and the absorbance value of each group of cells was analyzed in order to determine the ability of each lactic acid bacterium to remove 2,2-diphenyl-1-picrylhydrazyl (DPPH) free radicals, i.e., to calculate the DPPH free radical removal rate of each lactic acid bacterium, as shown in Table 2.

TABLE 1
Lactic acid bacterium samples and their sources

| Strain | | | |
|---|---|---|---|
| code | Bacterium species | Strain no. | Source |
| NB01 | Lactobacillus rhamnosus | LGG176 | Commercially available |
| | G.G. | | |
| NB03 | Pediococcus pentosaceus | PP365 | Commercially available |
| NB04 | Pediococcus pentosaceus | PP366 | Commercially available |
| NB05 | Lactobacillus rhamnosus | LCR103 | Commercially available |
| NB06 | Lactobacillus rhamnosus | LCR177 | BCRC910473 |
| NB07 | Lactobacillus plantarum | LP168 | Commercially available |
| NB08 | Lactobacillus plantarum | LP109 | BCRC910513 |
| NB09 | Lactobacillus plantarum | LP110 | BCRC910514 |
| NB10 | Lactobacillus plantarum | LP112 | BCRC911190 |
| NB11 | Lactobacillus plantarum | LP125 | Commercially available |
| NB12 | Lactobacillus plantarum | LP142 | Commercially available |
| NB13 | Lactobacillus plantarum | TWK10 | Commercially available |
| NB15 | Lactobacillus salivarius | LS159 | BCRC910700, |
| | | | CGMCC13316 |
| NB17 | Lactobacillus acidophilus | LA107 | Commercially available |
| NB18 | Lactobacillus paracasei | LPC188 | Commercially available |
| NB19 | Lactobacillus paracasei | LPC195 | Commercially available |
| NB20 | Lactobacillus paracasei | NTU101 | Commercially available |
| NB22 | Lactobacillus casei | LCC138 | Commercially available |
| NB23 | Lactobacillus paracasei | NB23 | BCRC911201 |
| NB24 | Lactobacillus fermentum | LF128 | Commercially available |
| NB25 | Bifidobacterium lactis | BLA281 | Commercially available |
| NB26 | Streptococcus | ST138 | Commercially available |
| | thermophilus | | |
| NB27 | Lactobacillus reuteri | LR107 | Commercially available |
| NB28 | Lactobacillus reuteri | BCRC14625 | Obtainable from the |
| | | | depository |
| NB29 | Lactobacillus reuteri | ATCC | Obtainable from the |
| | | (PTA-6475) | depository |
| NB30 | Lactococcus lactis | LL358 | BCRC910699, |
| | subsp. lactis | | CGMCC13317 |
| NB31 | Lactobacillus helveticus | LH163 | Commercially available |
| NB32 | Lactobacillus brevis | LBR109 | Commercially available |
| NB33 | Pediococcus acidilactici | PA318 | BCRC910474 |
| NB34 | Pediococcus acidilactici | PA319 | BCRC910515 |
| NB35 | Pediococcus acidilactici | PA320 | BCRC910516 |
| NB36 | Pediococcus acidilactici | PA325 | Commercially available |
| NB37 | Lactobacillus pentosus | LPE588 | Commercially available |
| NB38 | Bifidobacterium breve | BBr255 | Commercially available |
| NB40 | Bifidobacterium bifidum | BB223 | Commercially available |
| NB41 | Bifidobacterium longum | BL268 | Commercially available |
| NB42 | Bifidobacterium infantis | BIF217 | Commercially available |
| NB43 | Bifidobacterium | BCRC14607 | Obtainable from the |
| | adolescentis | | depository |
| NB44 | Bifidobacterium | BA286 | Commercially available |
| | adolescentis | | |
| NB45 | Lactobacillus gasseri | LG182 | Commercially available |
| NB47 | Lactobacillus delbrueckii | LB-87 | Commercially available |
| | subsp. bulgaricus | | |
| NB48 | Bacillus coagulans | BC208 | Commercially available |
| NB49 | Bacillus subtilis natto | BSN287 | Commercially available |

TABLE 2
DPPH free radical-removing ability of
each lactic acid bacterium strain

DPPH free radical removal rate (%)

| Strain | Diluted | Diluted | Diluted | Diluted |
|---|---|---|---|---|
| code | 8 times | 16 times | 50 times | 200 times |
| NB01 | 77.48 | 46.78 | 22.32 | 13.47 |
| NB03 | 82.45 | 51.89 | 25.51 | 19.55 |
| NB04 | 87.31 | 58.26 | 27.98 | 19.25 |
| NB05 | 79.09 | 58.35 | 28.84 | 23.59 |
| NB06 | 82.41 | 59.62 | 28.34 | 19 |
| NB07 | 80.75 | 57.53 | 32.61 | 26.52 |
| NB08 | 81.28 | 52.48 | 15.33 | 16.23 |
| NB09 | 76.39 | 53.84 | 29.74 | 23.66 |
| NB10 | 74.85 | 44.15 | 20.66 | 15.09 |
| NB11 | 70.46 | 40.61 | 21.81 | 11.08 |
| NB12 | 74.82 | 49.12 | 29.18 | 21.02 |
| NB13 | 76.57 | 55.91 | 27.69 | 16.41 |
| NB15 | 68.04 | 55.09 | 27.27 | 20.56 |
| NB17 | 88.18 | 71.82 | 35.03 | 22.87 |
| NB18 | 86.87 | 57.39 | 25.07 | 20.49 |
| NB19 | 81.44 | 60.6 | 30.54 | 26.1 |
| NB20 | 75.42 | 45.56 | 24.33 | 20.37 |
| NB22 | 68.87 | 53.07 | 28.28 | 18.57 |
| NB23 | 69.81 | 53.14 | 32.81 | 20.46 |
| NB24 | 68.23 | 41.91 | 22.21 | 15.89 |
| NB25 | 59.18 | 33.41 | 15.18 | 15.15 |
| NB26 | 66.9 | 48 | 29.74 | 27.2 |
| NB27 | 64.22 | 44.07 | 20.66 | 15.09 |
| NB28 | 62.13 | 38.84 | 31.51 | 25.29 |
| NB29 | 64.74 | 41.81 | 19.05 | 14.77 |
| NB30 | 74.77 | 44.44 | 21.39 | 16.66 |
| NB31 | 66.02 | 43.51 | 31.51 | 18.87 |
| NB32 | 60.61 | 46.73 | 32.7 | 20.57 |
| NB33 | 64.34 | 57.57 | 32.74 | 12.76 |
| NB34 | 58.64 | 41.5 | 35.58 | 27.71 |
| NB35 | 67.21 | 47.8 | 24.72 | 17.56 |
| NB36 | 78.02 | 51.66 | 22.01 | 19.06 |
| NB37 | 61.81 | 47.09 | 28.61 | 23.08 |
| NB38 | 85.01 | 58.59 | 32.33 | 26.15 |
| NB40 | 69.33 | 47.23 | 27.1 | 16.69 |
| NB41 | 69.19 | 40.76 | 16.92 | 8.91 |
| NB42 | 64.15 | 45.8 | 24.25 | 18.49 |
| NB43 | 81.57 | 45.58 | 28.95 | 20.87 |
| NB44 | 59.54 | 36.43 | 22.04 | 15.94 |
| NB45 | 69.98 | 49.51 | 31.67 | 23.99 |
| NB47 | 61.32 | 44.32 | 25.63 | 20.84 |
| NB48 | 41.26 | 35.86 | 22.91 | 18.91 |
| NB49 | 53.36 | 32.02 | 18.59 | 14.82 |

It can be known from the results in Table 2 that, generally speaking, the higher diluted times for a lactic acid bacterium, the lower DPPH free radical-removing ability to get; and that as far as each lactic acid bacterium is concerned, the sample with the dilution ratio of 1:8 had the strongest antioxidant effect, followed by the sample with the dilution ratio of 1:16.

More specifically, in all the bacterium samples with the dilution ratio of 1:8, strain code NB48 was the only strain whose DPPH free radical removal rate was lower than 50%, and the same strain had lower-than-40% DPPH free radical removal rates when diluted 50 times and 200 times. Moreover, in all the bacterium samples with the dilution ratio of 1:16, strain code NB17 had the highest DPPH free radical removal rate of 71.82%, and in all the bacterium samples with the dilution ratio of 1:50 or 1:200, the strains having the strongest DPPH free radical removal effects were strain codes NB34, NB17, and NB23, in descending order.

### EXAMPLE 3: TEST ON CELL SURVIVAL RATE

The lactic acid bacteria in Table 1 were obtained and cultured. Each cultured lactic acid bacterium was disrupted together with the supernatant collected from the corresponding culture and was subsequently diluted at different dilution ratios, i.e., diluted 16 times, 50 times, and 200 times, in order to produce differently diluted samples of each lactic acid bacterium. The C2C12 muscle cells cultured in example 1 were then treated separately with the lactic acid bacterium samples, and the survival rate of each group of C2C12 muscle cells that had been treated with a lactic acid bacterium sample of a specific dilution ratio was determined by an MTT assay. The test results are shown in Table 3.

TABLE 3
Survival rates of muscle cells treated with lactic
acid bacterium samples of different dilution ratios

| | Cell survival rate (%) | | | | | | | | |
|---|---|---|---|---|---|---|---|---|---|
| | Diluted 16 times | | | Diluted 50 times | | | Diluted 200 times | | |
| Strain | 24 | 48 | 72 | 24 | 48 | 72 | 24 | 48 | 72 |
| code | hours | hours | hours | hours | hours | hours | hours | hours | hours |
| NB01 | 71.93 | 52.58 | 56.34 | 96.86 | 53.26 | 45.98 | 101.99 | 86.55 | 94.45 |
| NB03 | 129.2 | 92.14 | 30.75 | 131.12 | 108.42 | 79.84 | 112.52 | 105.85 | 94.85 |
| NB04 | 131.73 | 102.34 | 117.56 | 126.17 | 114.99 | 120.27 | 108.74 | 95.51 | 106.15 |
| NB05 | 119.17 | 61.91 | 58.35 | 124.44 | 60.58 | 50.45 | 115.24 | 72.31 | 81.96 |
| NB06 | 99.48 | 60.48 | 69.09 | 109.55 | 52.86 | 70.16 | 100.18 | 61.18 | 91.36 |
| NB07 | 133.13 | 79.63 | 60.9 | 130.06 | 86.95 | 78.02 | 123.27 | 109.89 | 88.69 |
| NB08 | 113.61 | 101.45 | 25.8 | 126.66 | 109.28 | 68.07 | 100.29 | 115.94 | 96.3 |
| NB09 | 114.89 | 67.65 | 73.28 | 115.01 | 56.29 | 50.21 | 117.94 | 67.31 | 60.12 |
| NB10 | 106.34 | 73.44 | 124.16 | 109.75 | 77.97 | 93.94 | 106.68 | 69.58 | 92.2 |
| NB11 | 61.24 | 59.97 | 46.77 | 107.03 | 71.9 | 37.96 | 97.56 | 102.18 | 85.78 |
| NB12 | 92.32 | 63.85 | 77.26 | 99.03 | 52.22 | 72.07 | 98.45 | 71.84 | 87.83 |
| NB13 | 93.14 | 58.3 | 94.87 | 98.23 | 61.6 | 75.79 | 103.18 | 64.64 | 84.22 |
| NB15 | 125.87 | 77.58 | 31.74 | 119.38 | 100.25 | 85.24 | 111.67 | 114.34 | 96.58 |
| NB17 | 119.54 | 168.39 | 51.71 | 120.48 | 188.04 | 88.03 | 97.78 | 165.71 | 91.95 |
| NB18 | 123.73 | 85.27 | 125.39 | 122.93 | 71.59 | 98.87 | 121.12 | 80.23 | 98.42 |
| NB19 | 120.35 | 69.46 | 66.31 | 114.46 | 62.42 | 48.76 | 116.78 | 80.42 | 72.8 |
| NB20 | 123.3 | 101.11 | 126.39 | 122.44 | 98.65 | 96.16 | 133.25 | 77.57 | 109.8 |
| NB22 | 142.94 | 161.19 | 28.05 | 115.08 | 193.36 | 91.59 | 116.73 | 169.28 | 102.7 |
| NB23 | 145.04 | 93.92 | 61.67 | 89.79 | 96.09 | 79.86 | 84.1 | 96.75 | 90.79 |
| NB24 | 134.93 | 69.45 | 25.61 | 120.51 | 153.47 | 86.72 | 115.31 | 112.82 | 78.58 |
| NB25 | 86.44 | 65.15 | 48.44 | 100.8 | 63.88 | 41.65 | 100.06 | 89.38 | 85.41 |
| NB26 | 128.51 | 95.86 | 51.38 | 151.19 | 104.77 | 83.75 | 139.16 | 114.62 | 89.3 |
| NB27 | 80.94 | 75.12 | 75.41 | 82.41 | 102.8 | 90.45 | 95.08 | 95.3 | 80.99 |
| NB28 | 84.54 | 58.91 | 66.71 | 89.56 | 47.39 | 54.2 | 90.91 | 58.57 | 68.82 |
| NB29 | 107.66 | 92.42 | 83.74 | 110.39 | 106.3 | 98.72 | 102.08 | 94.57 | 90.58 |
| NB30 | 103.7 | 116.35 | 147.52 | 108.5 | 106.95 | 93.72 | 100.99 | 97.52 | 94.46 |
| NB31 | 130.17 | 72.68 | 25.58 | 115.55 | 108.82 | 92.64 | 109.21 | 98.59 | 97.3 |
| NB32 | 93.34 | 59 | 79.09 | 93.89 | 51.24 | 20.91 | 94.28 | 71.74 | 78.75 |
| NB33 | 61.12 | 58.04 | 58.17 | 100.41 | 57.69 | 42.07 | 97.56 | 90.67 | 97.49 |
| NB34 | 115.91 | 60.34 | 46.22 | 120.16 | 56.16 | 50.55 | 103.24 | 70.41 | 83.16 |
| NB35 | 108.36 | 95.55 | 112.74 | 143.88 | 105.83 | 57.46 | 129.8 | 116.6 | 85.08 |
| NB36 | 117.17 | 95.05 | 141.26 | 126.62 | 102.78 | 116.92 | 105.76 | 85.18 | 107.93 |
| NB37 | 89.09 | 67.35 | 74.86 | 97.56 | 52.38 | 60.36 | 96.32 | 60.4 | 71.31 |
| NB38 | 143.1 | 55.59 | 34.75 | 153.23 | 71.74 | 44.16 | 148.57 | 107.11 | 83.33 |
| NB40 | 124.18 | 107.37 | 59.08 | 134.87 | 108.67 | 78.5 | 118.21 | 120.31 | 97.94 |
| NB41 | 71.76 | 54.58 | 46.97 | 118.83 | 76.93 | 46.87 | 100.74 | 92.85 | 69.33 |
| NB42 | 98.6 | 74.26 | 124.28 | 100.06 | 105.32 | 90.89 | — | — | — |
| NB43 | 108.98 | 82.58 | 151.55 | 108.36 | 100.38 | 111.07 | 91.47 | 87.42 | 90.34 |
| NB44 | 81.68 | 53.83 | 52.52 | 112.46 | 61.35 | 50.61 | 102.07 | 81.59 | 85.79 |
| NB45 | 100.11 | 59.88 | 59.95 | 111.24 | 55.17 | 49.77 | 113.65 | 63.73 | 68.75 |
| NB47 | 91.7 | 79.77 | 97.43 | 87.29 | 105.95 | 98.31 | 75.16 | 74.73 | 68.37 |
| NB48 | 124.39 | 96.96 | 91.67 | 117.48 | 101.95 | 125.3 | 105.1 | 95.08 | 88.51 |
| NB49 | 129.8 | 151.23 | 49.81 | 125.27 | 172.52 | 96.13 | 109.29 | 93.18 | 100.54 |

It can be known from the results in Table 3 that the lactic acid bacterium samples with the dilution ratio of 1:16 had dose-dependent and time-dependent effects on the C2C12 muscle cells; that the higher the concentration of a lactic acid bacterium sample, the lower the survival rate of the muscle cells; and that the longer the treatment time, the lower the survival rate. While strain codes NB04, 10, 18, 20, 30, 36, 42, and 43 gave rise to survival rates higher than 100% when diluted 16 times and co-cultured with the muscle cells for 72 hours, it was found that these eight strains tended to form residues in the bottom portions of a 96-well microplate, and that the residues could not be removed by filtration, the main cause being that the cell membrane properties of these strains made the bacteria prone to bond to the muscle cells. In view of this, the survival rates of the eight strains had no value of reference, and the eight strains were excluded from the following tests.

### EXAMPLE 4: ANALYSIS OF PROTEIN EXPRESSION IN MUSCLE CELLS

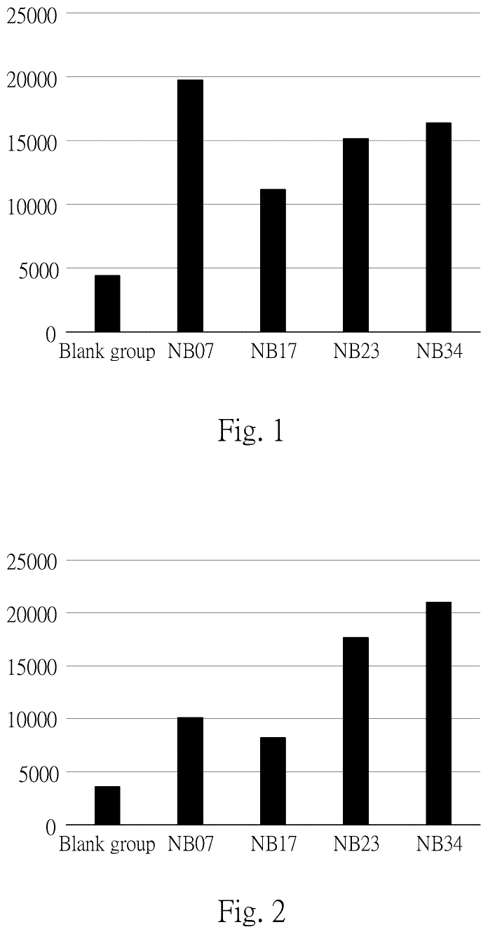

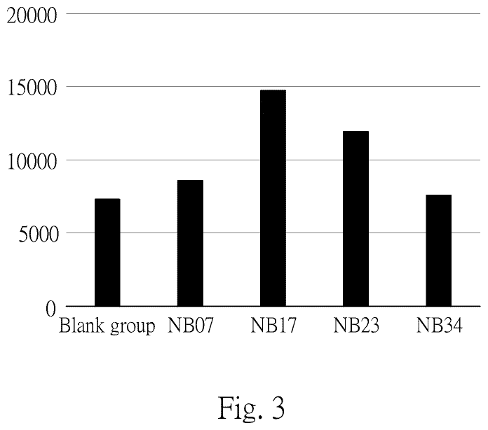

To select the strains to be used in this example, a screening process was performed based on the results in Table 2 and Table 3 and by considering the feasibility of quantification in relation to each strain. The selected lactic acid bacteria were strain codes NB07, NB17, NB23, and NB34. Each of these strains was co-cultured with the C2C12 muscle cells for 12 hours and 24 hours, and after co-culturing for 24 hours, the western blotting method was used to determine the protein content of each group of lactic acid bacterium-treated C2C12 muscle cells and the expression of PI3K, Akt, and mTOR proteins (which are related to muscle cell proliferation) in each group of cells. The results are shown in Table 4 and FIG. 1 to FIG. 3. The gene protein concentrations shown in FIG. 1 to FIG. 3 are values obtained by quantifying the western blot results (bands of different color intensities) with image processing software ImageJ.

TABLE 4
Protein contents of muscle cells co-cultured
with different lactic acid bacterium strains

| | 95% CI | | |
|---|---|---|---|
| Protein content of C2C12 | Upper | Lower | |
| muscle cells (mg/mL) | limit | limit | P value |

Co-culturing for 12 hours

| | | | | | |
|---|---|---|---|---|---|
| Blank group | 10.48 | (0.00382) | 9.84 | 11.13 | |
| NB07 | 5.14 | (0.00099) | 4.98 | 5.31 | 0.000057 |
| NB17 | 6.38 | (0.00127) | 6.16 | 6.59 | 0.000061 |
| NB23 | 11.41 | (0.00141) | 11.17 | 11.65 | 0.000024 |
| NB34 | 5.99 | (0.00049) | 5.90 | 6.07 | 0.000011 |

Co-culturing for 24 hours

| | | | | | |
|---|---|---|---|---|---|
| Blank group | 13.21 | (0.00290) | 12.72 | 13.70 | |
| NB07 | 13.6 2 | (0.00488) | 12.80 | 14.44 | 0.000198 |
| NB17 | 14.75 | (0.00276) | 14.29 | 15.22 | 0.000054 |
| NB23 | 13.86 | (0.00148) | 13.61 | 14.11 | 0.000018 |
| NB34 | 12.44 | (0.00042) | 12.37 | 12.51 | 0.000002 |

It can be known from the results in Table 4 and FIG. 1 to FIG. 3 that the lactic acid bacterium strain having the strongest effect of increasing muscle cell quantity was NB 23, followed sequentially by NB07, NB 34, and NB 17.

It can be inferred from the results of example 2 to example 4 that the Lactobacillus paracasei NB23 strain disclosed herein can remove the free radicals in muscle cells, maintain the proliferation and survival rate of muscle cells, encourage the proliferation and hypertrophy of muscle cells by enhancing the expression of PI3K and Akt proteins, and promote the synthesis and increase of muscles by increasing the expression of mTOR protein. In other words, the Lactobacillus paracasei NB23 strain disclosed herein has the activity to treat or improve insufficient muscle mass or diseases related thereto; that is to say, administering an effective amount of the Lactobacillus paracasei NB23 strain, a metabolite thereof, or a composition containing either of the above to an individual can effectively increase the individual's muscle mass and muscle strength, thereby producing the effect of treating or preventing sarcopenia and related symptoms.

### EXAMPLE 5: HUMAN SUBJECT RESEARCH

A plurality of human subjects were randomly divided into two groups, and during the 12-week research period:

Each subject in the placebo group was given 3 g of a placebo (maltodextrin starch) every day after dinner as well as after breakfast; and

Each subject in the NB23 group was given one sachet of a sample containing the Lactobacillus paracasei NB23 strain (3 g/sachet, the viable count per sachet being 50 billion CFU) every day after dinner as well as after breakfast.

### EXAMPLE 6: ANALYSIS OF BLOOD PRESSURES, BODY FAT PERCENTAGE, AND SKELETAL MUSCLE PERCENTAGES

Each group of subjects in example 5 went through the following tests:

In week 0 and week 12, the subjects in each group had their skeletal muscle percentages and body fat percentages measured with a commercially available body fat measuring device; and

In week 0 and week 12, a muscle strength/function evaluation was performed as follows: The muscle strength of the upper limbs was evaluated by testing the grip strength with a handheld grip strength tester (Dynamometer, TTM, Tokyo, Japan), and the muscle strength of the lower limbs was evaluated by counting the number of times of standing up from a chair and sitting down on the chair.

Analyses of the measurement/evaluation results are shown in Table 5 to Table 10.

TABLE 5
Analysis of the blood pressures, body fat percentages, and skeletal
muscle percentages of the subjects in each group in week 0

| | NB23 group | | Placebo group | |
|---|---|---|---|---|
| | | Stan- | | Stan- |
| | | dard | | dard |
| | | devi- | | devi- |
| | Mean | ation | Mean | ation |
| Age (years) | 38.26 | 9.65 | 35.96 | 10.79 |
| Body height (cm) | 164.50 | 9.17 | 162.06 | 7.88 |
| Systolic pressure (mmHg) | 120.33 | 15.01 | 118.48 | 14.62 |
| Diastolic pressure (mmHg) | 76.22 | 11.74 | 71.32 | 10.40 |
| Body weight (kg) | 69.67 | 18.00 | 64.37 | 11.70 |
| Body fat percentage (%) | 30.22 | 5.38 | 28.20 | 7.14 |
| BMI (kg/m²) | 25.70 | 4.92 | 24.26 | 4.06 |
| Subcutaneous fat - both legs (%) | 34.94 | 8.77 | 33.20 | 10.55 |
| Subcutaneous fat - whole body (%) | 25.21 | 6.57 | 23.91 | 8.00 |
| Subcutaneous fat - both wrists (%) | 37.16 | 10.12 | 36.46 | 11.90 |
| Subcutaneous fat - torso (%) | 21.78 | 5.90 | 20.59 | 7.14 |
| Average subcutaneous fat percentage | 29.77 | 7.61 | 28.54 | 9.30 |
| Skeletal muscle - both legs (%) | 40.98 | 4.93 | 41.63 | 5.88 |
| Skeletal muscle - whole body (%) | 26.88 | 3.13 | 27.60 | 4.04 |
| Skeletal muscle - both wrists (%) | 29.18 | 5.57 | 29.74 | 6.65 |
| Skeletal muscle - torso (%) | 20.62 | 2.89 | 21.63 | 4.24 |
| Average skeletal muscle percentage | 29.41 | 3.92 | 30.15 | 5.06 |
| Average grip strength (kg) | 32.88 | 9.83 | 29.89 | 7.49 |
| Number of times of stand-to-sit per | 30.48 | 6.61 | 31.60 | 5.70 |
| minute | | | | |

TABLE 6
Analysis of the blood pressures, body fat percentages, and skeletal
muscle percentages of the subjects in each group in week 12

| | NB23 group | | Placebo group | |
|---|---|---|---|---|
| | | Stan- | | Stan- |
| | | dard | | dard |
| | | devi- | | devi- |
| | Mean | ation | Mean | ation |
| Systolic pressure (mmHg) | 115.81 | 10.04 | 118.80 | 14.96 |
| Diastolic pressure (mmHg) | 72.41 | 10.22 | 71.79 | 10.22 |
| Body weight (kg) | 69.44 | 18.14 | 64.28 | 12.01 |
| Body fat percentage (%) | 25.51 | 5.11 | 24.40 | 4.34 |
| BMI (kg/m²) | 28.36 | 5.15 | 28.11 | 7.55 |
| Subcutaneous fat - both legs (%) | 33.27 | 7.22 | 32.220 | 10.89 |
| Subcutaneous fat - whole body (%) | 24.34 | 6.04 | 23.14 | 8.24 |
| Subcutaneous fat - both wrists (%) | 36.21 | 9.04 | 35.41 | 12.40 |
| Subcutaneous fat - torso (%) | 21.31 | 5.54 | 19.96 | 7.40 |
| Average subcutaneous fat percentage | 28.78 | 6.71 | 27.68 | 9.64 |
| Skeletal muscle - both legs (%) | 42.63 | 5.69 | 41.56 | 6.00 |
| Skeletal muscle - whole body (%) | 27.66 | 3.38 | 27.52 | 4.30 |
| Skeletal muscle - both wrists (%) | 29.96 | 5.79 | 29.52 | 6.70 |
| Skeletal muscle - torso (%) | 23.68 | 3.78 | 19.81 | 2.37 |
| Average skeletal muscle percentage | 30.98 | 3.68 | 29.60 | 4.53 |
| Average grip strength (kg) | 32.99 | 11.08 | 34.82 | 20.35 |
| Number of times of stand-to-sit per | 34.19 | 6.65 | 29.60 | 5.82 |
| minute | | | | |

TABLE 7
Analysis of the differences in blood pressures, body
fat percentage, and skeletal muscle percentages of
the subjects in each group between week 0 and week 12

| | NB23 group | | Placebo group | |
|---|---|---|---|---|
| | | Stan- | | Stan- |
| | | dard | | dard |
| | | devi- | | devi- |
| | Mean | ation | Mean | ation |
| Systolic pressure (mmHg) | −4.69 | 10.88 | 0.32 | 10.05 |
| Diastolic pressure (mmHg) | −3.96 | 5.12 | 0.44 | 5.37 |
| Body weight (kg) | −0.24 | 2.54 | −0.09 | 1.61 |
| Body fat percentage (%) | −1.93 | 1.96 | −0.09 | 1.24 |
| BMI (kg/m²) | −0.20 | 0.93 | 0.13 | 1.96 |
| Subcutaneous fat - both legs (%) | −1.73 | 3.88 | −1.01 | 4.35 |
| Subcutaneous fat - whole body (%) | −0.91 | 1.82 | −0.78 | 3.44 |
| Subcutaneous fat - both wrists (%) | −0.98 | 7.02 | −1.05 | 4.88 |
| Subcutaneous fat - torso (%) | −0.49 | 2.17 | −0.64 | 2.92 |
| Average subcutaneous fat percentage | −1.03 | 3.48 | −0.87 | 3.89 |
| Skeletal muscle - both legs (%) | 1.71 | 4.18 | −0.07 | 0.69 |
| Skeletal muscle - whole body (%) | 0.80 | 2.72 | −0.07 | 0.56 |
| Skeletal muscle - both wrists (%) | 0.81 | 1.75 | −0.22 | 0.91 |
| Skeletal muscle - torso (%) | 3.18 | 2.83 | −1.82 | 3.12 |
| Average skeletal muscle percentage | 1.63 | 1.86 | −0.55 | 0.85 |
| Average grip strength (kg) | 0.11 | 4.51 | 4.93 | 16.85 |
| Number of times of stand-to-sit per | 3.85 | 4.71 | −2.00 | 3.07 |
| minute | | | | |

TABLE 8
Analysis, by gender, of the differences in blood pressures, body fat percentage,
and skeletal muscle percentages of the subjects between week 0 and week 12

| | Male | | | | Female | | | |
|---|---|---|---|---|---|---|---|---|
| | NB23 group | | Placebo group | | NB23 group | | Placebo group | |
| | Mean | SD | Mean | SD | Mean | SD | Mean | SD |
| Systolic pressure (mmHg) | −12.63 | 11.60 | 4.57 | 5.06 | −1.17 | 8.72 | −1.33 | 11.10 |
| Diastolic pressure (mmHg) | −4.50 | 4.75 | 4.14 | 3.80 | −3.72 | 5.39 | −1.00 | 5.27 |
| Body weight (kg) | 0.78 | 3.23 | 0.17 | 0.99 | −0.69 | 2.10 | −0.19 | 1.50 |
| Body fat percentage (%) | −1.28 | 2.11 | −0.30 | 1.31 | −2.23 | 1.87 | −0.01 | 1.24 |
| BMI (kg/m²) | 0.11 | 0.69 | −0.14 | 0.52 | −0.33 | 1.01 | 0.24 | 2.30 |
| Subcutaneous fat - both legs | −1.06 | 4.50 | −0.63 | 1.41 | −2.02 | 3.68 | −1.16 | 5.09 |
| (%) | | | | | | | | |
| Subcutaneous fat - whole | −0.60 | 2.54 | −0.47 | 0.82 | −1.04 | 1.47 | −0.89 | 4.05 |
| body (%) | | | | | | | | |
| Subcutaneous fat - both | −1.80 | 6.53 | −0.54 | 1.27 | −0.62 | 7.39 | −1.24 | 5.73 |
| wrists (%) | | | | | | | | |
| Subcutaneous fat - torso (%) | −0.50 | 2.09 | −0.44 | 0.70 | −0.49 | 2.26 | −0.71 | 3.44 |
| Average subcutaneous fat | −0.99 | 3.91 | −0.52 | 1.05 | −1.04 | 3.39 | −1.00 | 4.57 |
| percentage | | | | | | | | |
| Skeletal muscle - both legs | 2.20 | 4.11 | 0.07 | 0.75 | 1.49 | 4.31 | −0.12 | 0.68 |
| (%) | | | | | | | | |
| Skeletal muscle - whole | 0.35 | 3.70 | 0.21 | 0.47 | 1.01 | 2.25 | −0.18 | 0.57 |
| body (%) | | | | | | | | |
| Skeletal muscle - both wrists | 1.10 | 2.84 | −0.31 | 0.94 | 0.68 | 1.06 | −0.19 | 0.92 |
| (%) | | | | | | | | |
| Skeletal muscle - torso (%) | 2.68 | 2.44 | −5.66 | 2.65 | 3.41 | 3.03 | −0.33 | 1.68 |
| Average skeletal muscle | 1.58 | 2.61 | −1.42 | 0.55 | 1.65 | 1.50 | −0.21 | 0.69 |
| percentage | | | | | | | | |
| Average grip strength (kg) | 1.56 | 6.51 | 15.42 | 30.28 | −0.53 | 3.32 | 0.85 | 3.79 |
| Number of times of stand-to- | 3.00 | 1.07 | −2.14 | 3.34 | 4.22 | 5.63 | −1.94 | 3.06 |
| sit per minute | | | | | | | | |

TABLE 9
Analysis, by age, of the differences in blood pressures, body fat percentage,
and skeletal muscle percentages of the subjects between week 0 and week 12

| | 20-40 years of age | | | | 40-60 years of age | | | |
|---|---|---|---|---|---|---|---|---|
| | NB23 group | | Placebo group | | NB23 group | | Placebo group | |
| | Mean | SD | Mean | SD | Mean | SD | Mean | SD |
| Systolic pressure (mmHg) | −3.15 | 8.99 | 1.94 | 8.75 | −6.23 | 12.68 | −3.86 | 12.60 |
| Diastolic pressure (mmHg) | −3.31 | 4.63 | 0.83 | 5.12 | −4.62 | 5.68 | −0.57 | 6.27 |
| Body weight (kg) | −0.38 | 3.12 | −0.07 | 1.53 | −0.11 | 1.89 | −0.13 | 1.93 |
| Body fat percentage (%) | −2.37 | 1.65 | −0.22 | 1.40 | −1.50 | 2.20 | 0.24 | 0.62 |
| BMI (kg/m²) | −0.22 | 1.16 | 0.21 | 2.29 | −0.18 | 0.69 | −0.06 | 0.70 |
| Subcutaneous fat - both legs | −2.61 | 4.18 | −1.30 | 5.08 | −0.85 | 3.50 | −0.26 | 1.33 |
| (%) | | | | | | | | |
| Subcutaneous fat - whole | −1.49 | 2.03 | −1.04 | 4.01 | −0.32 | 1.43 | −0.09 | 0.90 |
| body (%) | | | | | | | | |
| Subcutaneous fat - both | −2.90 | 5.53 | −1.35 | 5.73 | 0.93 | 8.01 | −0.27 | 1.13 |
| wrists (%) | | | | | | | | |
| Subcutaneous fat - torso (%) | −1.01 | 1.75 | −0.83 | 3.41 | 0.02 | 2.48 | −0.13 | 0.83 |
| Average subcutaneous fat | −2.00 | 3.26 | −1.13 | 4.55 | −0.05 | 3.54 | −0.19 | 1.03 |
| percentage | | | | | | | | |
| Skeletal muscle - both legs | 2.80 | 5.44 | −0.13 | 0.77 | 0.62 | 2.05 | 0.10 | 0.46 |
| (%) | | | | | | | | |
| Skeletal muscle - whole | 1.09 | 2.53 | −0.07 | 0.65 | 0.52 | 2.97 | −0.07 | 0.25 |
| body (%) | | | | | | | | |
| Skeletal muscle - both wrists | 1.12 | 2.23 | −0.15 | 0.89 | 0.49 | 1.09 | −0.41 | 1.01 |
| (%) | | | | | | | | |
| Skeletal muscle - torso (%) | 1.99 | 1.63 | −2.19 | 3.02 | 4.37 | 3.32 | −0.89 | 3.41 |
| Average skeletal muscle | 1.75 | 2.22 | −0.64 | 0.79 | 1.50 | 1.48 | −0.32 | 1.01 |
| percentage | | | | | | | | |
| Average grip strength (kg) | −0.08 | 4.00 | 1.90 | 3.59 | 0.31 | 5.13 | 12.72 | 31.64 |
| Number of times of stand-to- | 3.46 | 6.06 | −2.78 | 3.25 | 4.23 | 3.03 | 0.00 | 1.15 |
| sit per minute | | | | | | | | |

TABLE 10
Analysis, by BMI, of the differences in blood pressures, body fat percentage,
and skeletal muscle percentages of the subjects between week 0 and week 12

| | BMI < 24 kg/m² | | | | BMI ≥ 24 kg/m² | | | |
|---|---|---|---|---|---|---|---|---|
| | NB23 group | | Placebo group | | NB23 group | | Placebo group | |
| | Mean | SD | Mean | SD | Mean | SD | Mean | SD |
| Systolic pressure (mmHg) | −0.67 | 10.26 | −0.42 | 8.81 | −8.14 | 10.53 | 1.00 | 11.39 |
| Diastolic pressure (mmHg) | −5.25 | 5.83 | 0.17 | 6.04 | −2.86 | 4.33 | 0.69 | 4.91 |
| Body weight (kg) | −0.07 | 2.78 | −0.35 | 1.27 | −0.39 | 2.39 | 0.15 | 1.89 |
| Body fat percentage (%) | −1.84 | 1.50 | −0.37 | 1.50 | −2.01 | 2.34 | 0.16 | 0.83 |
| BMI (kg/m²) | −0.10 | 0.66 | −0.02 | 0.62 | −0.28 | 1.14 | 0.27 | 2.70 |
| Subcutaneous fat - both legs | −1.30 | 3.75 | −0.41 | 1.54 | −2.09 | 4.09 | −1.56 | 5.92 |
| (%) | | | | | | | | |
| Subcutaneous fat - whole | −0.94 | 2.12 | −0.22 | 1.09 | −0.88 | 1.60 | −1.29 | 4.68 |
| body (%) | | | | | | | | |
| Subcutaneous fat - both | 0.00 | 9.48 | −0.36 | 1.64 | −1.83 | 4.14 | −1.68 | 6.65 |
| wrists (%) | | | | | | | | |
| Subcutaneous fat - torso (%) | −0.17 | 2.70 | −0.20 | 1.15 | −0.77 | 1.64 | −1.04 | 3.93 |
| Average subcutaneous fat | −0.60 | 4.27 | −0.30 | 1.33 | −1.39 | 2.75 | −1.39 | 5.29 |
| percentage | | | | | | | | |
| Skeletal muscle - both legs | 1.43 | 3.93 | −0.06 | 0.78 | 1.96 | 4.51 | −0.08 | 0.63 |
| (%) | | | | | | | | |
| Skeletal muscle - whole | 1.26 | 2.93 | 0.02 | 0.64 | 0.41 | 2.56 | −0.15 | 0.49 |
| body (%) | | | | | | | | |
| Skeletal muscle - both wrists | 1.06 | 2.32 | −0.16 | 1.06 | 0.59 | 1.12 | −0.28 | 0.78 |
| (%) | | | | | | | | |
| Skeletal muscle - torso (%) | 3.59 | 3.12 | −2.90 | 3.33 | 2.83 | 2.63 | −0.83 | 2.65 |
| Average skeletal muscle | 1.83 | 1.97 | −0.78 | 0.93 | 1.45 | 1.80 | −0.34 | 0.74 |
| percentage | | | | | | | | |
| Average grip strength (kg) | −1.77 | 3.76 | 1.48 | 3.42 | 1.73 | 4.59 | 8.11 | 23.12 |
| Number of times of stand-to- | 4.67 | 4.27 | −2.33 | 3.77 | 3.14 | 5.11 | −1.69 | 2.36 |
| sit per minute | | | | | | | | |

It can be known from the results in Table 5 and Table 6 that in week 0 of the test, there was no difference in body fat percentage or skeletal muscle percentages between the two groups of subjects; and that in week 12 of the test, skeletal muscle-torso and the number of times of stand-to-sit per minute of the NB23 group of subjects were significantly higher than those of the placebo group. It can be further known from the results in Table 7 that the NB23 group of subjects showed significantly greater improvements in the following items than the placebo group: diastolic pressure, body fat percentage, skeletal muscle percentage-both legs, skeletal muscle percentage-both wrists, skeletal muscle percentage
torso, average skeletal muscle percentage, and the number of times of stand-to-sit per minute. More specifically, the NB23 group showed a significant increase in skeletal muscle percentage-both legs, skeletal muscle percentage-both wrists, skeletal muscle percentage
torso, average skeletal muscle percentage, and the number of times of stand-to-sit per minute while the placebo group showed a decrease in each of the aforesaid items; and the NB23 group showed a decrease in diastolic pressure and body fat percentage while the placebo group showed either an increase or a relatively small decrease in each of the aforesaid items.

It can be known from the results in Table 8 that the female subjects in the NB23 group showed an increase in skeletal muscle percentage-whole body, skeletal muscle percentage-both wrists, skeletal muscle percentage-torso, average skeletal muscle percentage, and the number of times of stand-to-sit per minute while their counterparts in the placebo group showed a decrease in each of the aforesaid items; that the female subjects in the NB23 group showed a significantly greater decrease in body fat percentage than their counterparts in the placebo group; and that the male subjects in the NB23 group showed a greater decrease in diastolic pressure than their female counterparts in the same group.

It can be known from the results in Table 9 that in the NB23 group, the subjects aged 40-60 showed a significantly greater increase in skeletal muscle percentage-torso than the subjects aged 20-40. It can be known from the results in Table 10 that the subjects with BMI<24 kg/m2 in the NB23 group showed a significant increase in skeletal muscle percentage-torso, average skeletal muscle percentage, and the number of times of stand-to-sit per minute while the subjects with BMI<24 kg/m2 in the placebo group showed a decrease in each of the aforesaid items; that the subjects with BMI<24 kg/m2 in the NB23 group showed a significant decrease in diastolic pressure and body fat percentage while the subjects with BMI<24 kg/m2 in the placebo group showed either an increase or a relatively small decrease in each of the aforesaid items; that the subjects with BMI<24 kg/m2 in the NB23 group showed a significant decrease in average grip strength while the subjects with BMI<24 kg/m2 in the placebo group showed an increase; and that in the NB23 group, the subjects with BMI≥24 kg/m2 showed an increase in average grip strength while the subjects with BMI<24 kg/m2 showed a decrease.

### EXAMPLE 7: DETERMINATION AND ANALYSIS OF THE BIOCHEMICAL VALUES OF BLOOD

In week 0 and week 12 of the test, the subjects in each group of example 5 had a biochemical test on their blood, and the results are shown in Table 11 to Table 15, wherein TG stands for triglycerides, TC stands for total cholesterol, LDL-C stands for low-density lipoprotein cholesterol, HDL-C stands for high-density lipoprotein cholesterol, BUN stands for blood urea nitrogen, GOT stands for serum glutamic oxaloacetic transaminase (aspartate transaminase), GPT stands for serum glutamate-pyruvate transaminase (alanine transaminase), TP stands for total protein, CRP stands for C-reactive protein, and LDH stands for lactate dehydrogenase.

TABLE 11
Analysis of biochemical values of blood of subjects
in each group in week 0 and week 12

| | Week 0 | | | | Week 12 | | | |
|---|---|---|---|---|---|---|---|---|
| | NB23 group | | Placebo group | | NB23 group | | Placebo group | |
| | Mean | SD | Mean | SD | Mean | SD | Mean | SD |
| TG (mg/dL) | 135.85 | 44.73 | 123.24 | 39.30 | 118.42 | 42.34 | 142.08 | 46.14 |
| TC (mg/dL) | 211.56 | 30.86 | 197.84 | 28.68 | 194.23 | 31.55 | 202.48 | 29.59 |
| LDL-C (mg/dL) | 120.44 | 30.62 | 109.08 | 23.21 | 100.69 | 22.17 | 119.12 | 26.28 |
| HDL-C (mg/dL) | 57.19 | 15.17 | 61.68 | 16.80 | 59.88 | 14.33 | 60.52 | 13.32 |
| TC/HDL-C | 3.95 | 1.15 | 3.45 | 1.09 | 3.47 | 1.21 | 3.48 | 0.81 |
| LDL-C/HDL-C | 2.31 | 1.11 | 1.95 | 0.82 | 1.79 | 0.62 | 2.09 | 0.72 |
| BUN (mg/dL) | 12.59 | 3.20 | 12.24 | 3.27 | 10.69 | 2.91 | 13.44 | 4.84 |
| Creatinine (mg/dL) | 0.83 | 0.19 | 0.78 | 0.20 | 0.76 | 0.23 | 0.80 | 0.20 |
| Uric acid (mg/dL) | 5.74 | 1.54 | 5.13 | 1.09 | 4.67 | 1.13 | 5.08 | 0.79 |
| GOT (U/dL) | 21.26 | 4.39 | 19.80 | 4.11 | 18.08 | 4.02 | 19.84 | 4.31 |
| GPT (U/dL) | 22.52 | 11.16 | 19.12 | 5.53 | 20.00 | 9.09 | 18.28 | 7.10 |
| TP (g/dL) | 7.25 | 0.84 | 7.22 | 0.60 | 7.06 | 0.81 | 7.33 | 0.43 |
| Albumin (g/dL) | 4.75 | 0.70 | 4.68 | 0.29 | 4.78 | 0.46 | 4.81 | 0.29 |
| Fasting blood | 99.20 | 9.21 | 99.06 | 7.42 | 96.81 | 11.26 | 100.40 | 8.08 |
| glucose (mg/dL) | | | | | | | | |
| HbA1C (%) | 5.55 | 0.38 | 5.53 | 0.29 | 5.47 | 0.37 | 5.57 | 0.27 |
| Insulin (U/dL) | 11.06 | 7.69 | 9.87 | 7.48 | 9.45 | 6.59 | 11.11 | 8.28 |
| Fibrinogen (mg/dL) | 247.64 | 42.33 | 257.38 | 50.20 | 256.49 | 49.54 | 232.79 | 51.96 |
| CRP (μg/dL) | 0.09 | 0.18 | 0.17 | 0.34 | 0.06 | 0.10 | 0.13 | 0.32 |
| LDH (mg/dL) | 171.07 | 30.57 | 159.00 | 24.36 | 201.00 | 239.48 | 165.20 | 20.68 |
| Vitamin D (mg/dL) | 21.03 | 8.13 | 23.26 | 5.99 | 24.65 | 7.80 | 24.02 | 5.64 |

TABLE 12
Analysis of the differences of the biochemical values
of blood after consumption of the lactic acid bacterium

| | |
|---|---|
| NB23 group | Placebo group |

| | Mean | SD | Mean | SD |
|---|---|---|---|---|
| TG (mg/dL) | −17.85 | 32.64 | 18.84 | 30.86 |
| TC (mg/dL) | −18.54 | 14.15 | 4.64 | 12.00 |
| LDL-C (mg/dL) | −20.19 | 19.79 | 10.04 | 16.18 |
| HDL-C (mg/dL) | 2.08 | 9.83 | −1.16 | 7.79 |
| TC/HDL-C | −0.46 | 1.12 | 0.04 | 0.54 |
| LDL-C/HDL-C | −0.50 | 0.84 | 0.14 | 0.30 |
| BUN (mg/dL) | −1.85 | 3.13 | 1.20 | 4.89 |
| Creatinine (mg/dL) | −0.07 | 0.10 | 0.03 | 0.06 |
| Uric acid (mg/dL) | −1.01 | 1.15 | −0.05 | 0.92 |
| GOT (U/dL) | −2.81 | 4.15 | 0.04 | 3.55 |
| GPT (U/dL) | −1.77 | 4.13 | −0.84 | 5.07 |
| TP (g/dL) | −0.18 | 1.26 | 0.11 | 0.49 |
| Albumin (g/dL) | 0.04 | 0.52 | 0.12 | 0.16 |
| Fasting blood | −2.10 | 7.28 | 1.34 | 4.59 |
| glucose (mg/dL) | | | | |
| HbA1C (%) | −0.08 | 0.13 | 0.04 | 0.20 |
| Insulin (U/dL) | −1.45 | 2.99 | 1.24 | 4.35 |
| Fibrinogen (mg/dL) | 7.10 | 52.17 | −24.59 | 51.99 |
| CRP (μg/dL) | −0.03 | 0.14 | −0.03 | 0.29 |
| LDH (mg/dL) | 32.88 | 241.05 | 6.20 | 20.94 |
| Vitamin D (mg/dL) | 3.58 | 4.59 | 0.76 | 3.91 |

TABLE 13
Analysis, by gender, of the differences of the
biochemical values of the subjects' blood

| | Male | | | | Female | | | |
|---|---|---|---|---|---|---|---|---|
| | NB23 group | | Placebo group | | NB23 group | | Placebo group | |
| | Mean | SD | Mean | SD | Mean | SD | Mean | SD |
| TG (mg/dL) | −35.25 | 31.15 | 10.43 | 13.05 | −10.11 | 31.00 | 22.11 | 35.27 |
| TC (mg/dL) | −17.13 | 12.52 | 5.00 | 10.94 | −19.17 | 15.12 | 4.50 | 12.68 |
| LDL-C (mg/dL) | −21.50 | 23.25 | 10.29 | 9.67 | −19.61 | 18.76 | 9.94 | 18.34 |
| HDL-C (mg/dL) | 1.50 | 11.59 | −0.86 | 10.91 | 2.33 | 9.31 | −1.28 | 6.66 |
| TC/HDL-C | −0.44 | 1.30 | 0.00 | 0.75 | −0.47 | 1.07 | 0.05 | 0.46 |
| LDL-C/HDL-C | −0.51 | 0.42 | 0.13 | 0.37 | −0.50 | 0.98 | 0.14 | 0.29 |
| BUN (mg/dL) | −2.63 | 3.02 | 2.57 | 6.27 | −1.50 | 3.20 | 0.67 | 4.33 |
| Creatinine (mg/dL) | −0.04 | 0.07 | 0.03 | 0.05 | −0.08 | 0.11 | 0.02 | 0.07 |
| Uric acid (mg/dL) | −1.23 | 1.04 | −0.11 | 0.52 | −0.92 | 1.21 | −0.02 | 1.05 |
| GOT (U/dL) | −5.00 | 5.50 | 2.29 | 3.68 | −1.83 | 3.09 | −0.83 | 3.19 |
| GPT (U/dL) | −4.00 | 6.35 | 2.71 | 7.06 | −0.78 | 2.26 | −2.22 | 3.39 |
| TP (g/dL) | −0.26 | 1.36 | 0.11 | 0.57 | −0.15 | 1.25 | 0.11 | 0.48 |
| Albumin (g/dL) | −0.13 | 0.18 | 0.06 | 0.13 | 0.12 | 0.61 | 0.15 | 0.17 |
| Fasting blood | 0.38 | 9.81 | 0.57 | 3.05 | −3.19 | 5.84 | 1.64 | 5.11 |
| glucose (mg/dL) | | | | | | | | |
| HbA1C (%) | −0.09 | 0.16 | 0.07 | 0.28 | −0.08 | 0.12 | 0.02 | 0.17 |
| Insulin (U/dL) | −1.20 | 3.62 | 2.22 | 2.26 | −1.57 | 2.78 | 0.86 | 4.94 |
| Fibrinogen (mg/dL) | 4.90 | 62.41 | −34.10 | 55.95 | 8.08 | 48.94 | −20.89 | 51.57 |
| CRP (μg/dL) | −0.05 | 0.05 | 0.06 | 0.14 | −0.03 | 0.17 | −0.07 | 0.32 |
| LDH (mg/dL) | −22.63 | 17.25 | 22.29 | 21.73 | 57.56 | 288.50 | −0.06 | 17.45 |
| Vitamin D (mg/dL) | 4.09 | 6.11 | −0.13 | 3.71 | 3.36 | 3.93 | 1.10 | 4.04 |

TABLE 14
Analysis, by age, of the differences of the biochemical values of the subjects' blood

| | 20-40 years of age | | | | 40-60 years of age | | | |
|---|---|---|---|---|---|---|---|---|
| | NB23 group | | Placebo group | | NB23 group | | Placebo group | |
| | Mean | SD | Mean | SD | Mean | SD | Mean | SD |
| TG (mg/dL) | −22.85 | 37.58 | 13.28 | 19.43 | −12.85 | 27.44 | 33.14 | 49.07 |
| TC (mg/dL) | −22.23 | 16.89 | 4.22 | 11.82 | −14.85 | 10.12 | 5.71 | 13.33 |
| LDL-C (mg/dL) | −15.46 | 17.54 | 8.06 | 12.61 | −24.92 | 21.43 | 15.14 | 23.53 |
| HDL-C (mg/dL) | 4.38 | 7.35 | −0.44 | 7.18 | −0.23 | 11.66 | −3.00 | 9.54 |
| TC/HDL-C | −9.84 | 11.25 | −0.73 | 3.98 | −0.09 | 1.25 | 0.11 | 0.69 |
| LDL-C/HDL-C | −5.11 | 7.28 | 0.55 | 4.03 | −0.38 | 0.46 | 0.27 | 0.38 |
| BUN (mg/dL) | −1.15 | 2.15 | 0.72 | 3.92 | −2.54 | 3.84 | 2.43 | 7.04 |
| Creatinine (mg/dL) | −0.07 | 0.08 | 0.04 | 0.06 | −0.07 | 0.12 | 0.00 | 0.07 |
| Uric acid (mg/dL) | −1.27 | 1.24 | −0.14 | 1.00 | −0.75 | 1.03 | 0.19 | 0.69 |
| GOT (U/dL) | −1.92 | 2.87 | −0.11 | 3.39 | −3.69 | 5.09 | 0.43 | 4.20 |
| GPT (U/dL) | −0.92 | 3.30 | −0.72 | 5.52 | −2.62 | 4.81 | −1.14 | 4.06 |
| TP (g/dL) | 0.30 | 1.03 | 0.13 | 0.43 | −0.67 | 1.31 | 0.06 | 0.67 |
| Albumin (g/dL) | 0.12 | 0.70 | 0.13 | 0.17 | −0.04 | 0.25 | 0.10 | 0.13 |
| Fasting blood | −3.00 | 6.23 | 1.22 | 4.95 | −1.19 | 8.35 | 1.64 | 3.82 |
| glucose (mg/dL) | | | | | | | | |
| HbA1C (%) | −0.05 | 0.10 | 0.04 | 0.22 | −0.11 | 0.15 | 0.03 | 0.17 |
| Insulin (U/dL) | −1.43 | 3.18 | 1.39 | 4.90 | −1.48 | 2.93 | 0.85 | 2.71 |
| Fibrinogen (mg/dL) | 19.25 | 59.59 | −23.86 | 56.33 | −5.05 | 42.42 | −26.47 | 42.63 |
| CRP (μg/dL) | −0.07 | 0.18 | −0.09 | 0.30 | 0.00 | 0.07 | 0.11 | 0.19 |
| LDH (mg/dL) | 80.46 | 340.34 | 3.56 | 13.05 | −14.69 | 17.94 | 13.00 | 34.59 |
| Vitamin D (mg/dL) | 3.26 | 3.95 | 0.79 | 4.03 | 3.90 | 5.30 | 0.67 | 3.89 |

TABLE 15
Analysis, by BMI, of the differences of the
biochemical values of the subjects' blood

| | BMI < 24 kg/m² | | | | BMI ≥ 24 kg/m² | | | |
|---|---|---|---|---|---|---|---|---|
| | NB23 group | | Placebo group | | NB23 group | | Placebo group | |
| | Mean | SD | Mean | SD | Mean | SD | Mean | SD |
| TG (mg/dL) | −15.75 | 35.65 | 6.50 | 13.60 | −19.64 | 31.08 | 30.23 | 37.98 |
| TC (mg/dL) | −20.92 | 16.38 | 6.08 | 13.05 | −16.50 | 12.19 | 3.31 | 11.30 |
| LDL-C (mg/dL) | −14.67 | 15.56 | 8.50 | 8.79 | −24.93 | 22.27 | 11.46 | 21.17 |
| HDL-C (mg/dL) | 1.17 | 5.17 | −3.17 | 7.26 | 2.86 | 12.73 | 0.69 | 8.08 |
| TC/HDL-C | −0.39 | 0.38 | 0.21 | 0.38 | −0.52 | 1.51 | −0.13 | 0.62 |
| LDL-C/HDL-C | −0.25 | 0.30 | 0.17 | 0.24 | −0.72 | 1.08 | 0.11 | 0.36 |
| BUN (mg/dL) | −1.33 | 3.39 | 0.92 | 4.10 | −2.29 | 2.95 | 1.46 | 5.68 |
| Creatinine (mg/dL) | −0.07 | 0.12 | 0.04 | 0.06 | −0.06 | 0.09 | 0.01 | 0.07 |
| Uric acid (mg/dL) | −1.05 | 1.26 | −0.28 | 1.12 | −0.98 | 1.09 | 0.17 | 0.67 |
| GOT (U/dL) | −1.58 | 3.00 | 0.08 | 4.36 | −3.86 | 4.79 | 0.00 | 2.80 |
| GPT (U/dL) | −1.25 | 3.19 | −0.67 | 6.91 | −2.21 | 4.87 | −1.00 | 2.77 |
| TP (g/dL) | −0.13 | 0.84 | 0.05 | 0.31 | −0.24 | 1.56 | 0.16 | 0.63 |
| Albumin (g/dL) | 0.22 | 0.71 | 0.08 | 0.16 | −0.11 | 0.20 | 0.16 | 0.16 |
| Fasting blood | −2.33 | 6.71 | 2.42 | 5.35 | −1.89 | 7.98 | 0.35 | 3.69 |
| glucose (mg/dL) | | | | | | | | |
| HbA1C (%) | −0.08 | 0.14 | 0.07 | 0.21 | −0.09 | 0.12 | 0.01 | 0.20 |
| Insulin (U/dL) | −0.08 | 2.45 | 0.38 | 2.83 | −2.63 | 2.99 | 2.03 | 5.40 |
| Fibrinogen (mg/dL) | 4.88 | 50.71 | −6.28 | 44.35 | 9.01 | 55.22 | −41.50 | 54.39 |
| CRP (μg/dL) | −0.01 | 0.03 | −0.02 | 0.33 | −0.06 | 0.19 | −0.04 | 0.25 |
| LDH (mg/dL) | 86.08 | 354.75 | 5.00 | 16.13 | −12.71 | 20.18 | 7.31 | 25.21 |
| Vitamin D (mg/dL) | 3.49 | 1.67 | −0.10 | 3.68 | 3.66 | 6.17 | 1.55 | 4.10 |

It can be known from the results in Table 11 that the cardiovascular sclerosis indicators/factors (i.e., TC, LDL-C, and LDL-C/HDL-C), kidney function indicators/factors (i.e., BUN and uric acid), and myocardial infarction/liver function/muscle damage factor (i.e., GOT) of the subjects in the NB23 group had significantly lower values in week 12 than in week 0 of the test. It can be known from the results in Table 12 that the subjects in the NB23 group showed a significantly greater increase in vitamin D in blood than their counterparts in the placebo group, and that the subjects in the NB23 group showed a significant decrease in the following indicators while their counterparts in the placebo group showed either an increase or a relatively small decrease in each of the following indicators: TG, TC, LDL-C, TC/HDL-C, LDL-C/HDL-C, BUN, creatinine, uric acid, GOT, fasting blood glucose, HbAlC and insulin.

The results in Table 11 show that after administering the Lactobacillus paracasei NB23 strain for 12 weeks, the TG, LDL-C, and BUN values of the subjects in the NB23 group were significantly lower than those of the subjects in the placebo group. It can be further known from the results in Table 12 that after administering the Lactobacillus paracasei NB23 strain for 12 weeks, the cardiovascular sclerosis indicators/factors (i.e., serum TC, LDL-C, and LDL-C/HDL-C), kidney function indicators/factors (i.e., BUN and uric acid), and myocardial infarction/liver function/muscle damage factor (i.e., GOT) of the subjects in the NB23 group had significantly lower values than before the test (i.e., in week 0); and that none of the indicators, i.e., the biochemical values of blood, of the placebo group showed a significant difference after the test.

It can be known from the results in Table 13 that after taking the Lactobacillus paracasei NB23 strain for 12 weeks, the male subjects in the NB23 group showed a significant decrease in serum TG, TC, LDL-C, LDL-C/HDL-C, LDH, serum creatinine, uric acid, GOT, albumin, and insulin whereas their counterparts in the placebo group showed either an increase or a relatively small decrease in each of the aforesaid items; and that the female subjects in the NB23 group showed a significant decrease in TC, LDL-C, LDL-C/HDL-C, uric acid, TG, and HbAlc while their counterparts in the placebo group showed either an increase or a relatively small decrease in each of the aforesaid items.

It can be known from the results in Table 14 that the subjects aged 20-40 in the NB23 group showed a significant decrease in serum TC, LDL-C, LDL-C/HDL-C, and serum creatinine while their counterparts in the placebo group showed an increase in each of the aforesaid items. Moreover, after taking the Lactobacillus paracasei NB23 strain for 12 weeks, the subjects aged 20-40 in the NB23 group showed a significant decrease in serum TG, TC/HDL-C, serum uric acid, fasting blood glucose, and insulin whereas their counterparts in the placebo group showed either an increase or a relatively small decrease in each of the aforesaid items. The subjects aged 20-40 in the NB23 group also showed an increase in serum fibrinogen while their counterparts in the placebo group showed a decrease. In addition, the subjects aged 40-60 in the NB23 group showed a significant decrease in serum LDL-C, uric acid, TG, TC, and LDL-C/HDL-C while their counterparts in the placebo group showed an increase in each of the aforesaid items. Furthermore, the subjects aged 20-40 in the NB23 group showed a significantly greater decrease in such cardiovascular sclerosis factors as serum TC/HDL-C and LDL-C/HDL-C than the subjects aged 40-60 in the NB23 group and also showed a significant increase in serum TP while the subjects aged 40-60 in the NB23 group showed a decrease in the same item.

It can be known from the results in Table 15 that after taking the Lactobacillus paracasei NB23 strain for 12 weeks, the subjects with BMI<24 kg/m2 in the NB23 group showed a significant decrease in serum TC, LDL-C, TC/HDL-C, LDL-C/HDL-C, serum creatinine, and HbAlC while their counterparts in the placebo group showed an increase in each of the aforesaid items. Also, the subjects with BMI<24 kg/m2 in the NB23 group showed a significant increase in serum vitamin D while their counterparts in the placebo group showed a decrease. In addition, the subjects with BMI≥24 kg/m2 in the NB23 group showed a significant decrease in serum TG, TC, LDL-C, LDL-C/HDL-C, BUN, uric acid, and albumin while their counterparts in the placebo group showed an increase in each of the aforesaid items, and the subjects with BMI≥24 kg/m2 in the NB23 group showed an increase in serum fibrinogen while their counterparts in the placebo group showed a decrease.

It can be inferred from the foregoing results that administering the disclosed Lactobacillus paracasei NB23 strain continuously for 12 weeks can reduce the expression of LDH (an inflammation and cell damage indicator) in a subject, lower the values of cardiovascular sclerosis indicators/factors (i.e., LDL-C, TC/HDL-C, and LDL-C/HDL-C), and improve cardiovascular disease indicators (i.e., HDL-C and vitamin D) and metabolic syndrome indicators (by lowering the systolic pressure, reducing triglycerides and fasting blood glucose, and increasing HDL-C). In other words, the Lactobacillus paracasei NB23 strain disclosed herein or a metabolite thereof can be used to treat or prevent metabolic syndrome and cardiovascular-related diseases.

### EXAMPLE 8: ANALYSIS OF MUSCLE PROTEIN SYNTHESIS/DEGRADATION REGULATING FACTORS AND CELL DIFFERENTIATION AND MATURATION FACTOR

In week 0 and week 12, the following indicators of each subject in example 5 were tested: inflammation indictors TNF-α, IL-6, and poly (ADP-ribose) polymerase (PARP); cell protein degradation indicator NF-κB, Fox-O, and myoblast determination protein 1 (MyoD) (a cell differentiation and maturation factor); and cell protein synthesis indicators PI3K, Akt, mTOR (a myogenic regulatory factor), insulin, and IGF-1. The test results are shown in Table 16 to Table 20.

TABLE 16
Analysis of the muscle protein synthesis/degradation regulating
factors and cell differentiation and maturation factor in
the subjects in each group in week 0 and week 12

| | Week 0 | | | | Week 12 | | | |
|---|---|---|---|---|---|---|---|---|
| | NB23 group | | Placebo group | | NB23 group | | Placebo group | |
| | Mean | SD | Mean | SD | Mean | SD | Mean | SD |
| TNFα (pg/dL) | 225.71 | 128.43 | 193.89 | 98.45 | 33.10 | 32.93 | 202.51 | 102.41 |
| IL-6 (pg/dL) | 2.07 | 2.15 | 1.91 | 2.26 | 1.13 | 1.78 | 3.88 | 7.25 |
| PARP (pg/dL) | 28.61 | 15.94 | 21.90 | 10.44 | 22.02 | 20.89 | 18.36 | 7.60 |
| NF-κB (pg/dL) | 1.31 | 0.54 | 2.50 | 3.02 | 0.99 | 0.50 | 1.86 | 1.31 |
| Fox-O (pg/dL) | 2.34 | 2.68 | 3.95 | 4.93 | 1.34 | 1.93 | 4.24 | 6.40 |
| Insulin (U/dL) | 11.06 | 7.69 | 9.87 | 7.48 | 9.45 | 6.59 | 11.11 | 8.28 |
| MyoD (pg/dL) | 21.32 | 5.79 | 17.10 | 9.49 | 23.00 | 7.88 | 13.50 | 6.88 |
| PI3K (pg/dL) | 2.05 | 2.49 | 2.57 | 2.65 | 3.24 | 2.58 | 5.37 | 6.24 |
| mTOR (pg/dL) | 1.50 | 0.44 | 1.99 | 1.50 | 10.07 | 4.21 | 3.13 | 3.02 |
| IGF-1 (pg/dL) | 3.96 | 7.01 | 8.06 | 8.46 | 5.41 | 9.68 | 3.90 | 5.80 |
| pAkt (pg/dL) | 1144.91 | 795.99 | 1107.61 | 1111.18 | 1500.00 | 872.71 | 1353.35 | 1137.90 |

TABLE 17
Analysis of the differences of the muscle protein synthesis/degradation
regulating factors and cell differentiation and maturation factor
in the subjects in each group after the test

| | |
|---|---|
| NB23 group | Placebo group |

| | Mean | SD | Mean | SD |
|---|---|---|---|---|
| TNFα (pg/dL) | −194.45 | 130.79 | 4.83 | 22.94 |
| IL-6 (pg/dL) | −0.96 | 1.41 | 1.89 | 6.99 |
| PARP (pg/dL) | −11.56 | 16.92 | −3.54 | 11.44 |
| NF-κB (pg/dL) | −0.30 | 0.52 | −0.65 | 3.06 |
| Fox-O (pg/dL) | −0.82 | 2.06 | 0.13 | 5.99 |
| Insulin (U/dL) | −1.45 | 2.99 | 1.24 | 4.35 |
| MyoD (pg/dL) | 2.02 | 4.94 | −3.60 | 9.81 |
| PI3K (pg/dL) | 1.22 | 0.89 | 2.37 | 5.57 |
| mTOR (pg/dL) | 8.59 | 4.30 | 1.14 | 3.32 |
| IGF-1 (pg/dL) | 1.58 | 3.28 | −3.67 | 11.06 |
| pAkt (pg/dL) | 321.01 | 314.15 | 245.74 | 359.50 |

TABLE 18
Analysis, by gender, of the differences of the subjects' muscle protein synthesis/degradation
regulating factors and cell differentiation and maturation factor after the test

| | Male | | | | Female | | | |
|---|---|---|---|---|---|---|---|---|
| | NB23 group | | Placebo group | | NB23 group | | Placebo group | |
| | Mean | SD | Mean | SD | Mean | SD | Mean | SD |
| TNFα (pg/dL) | −127.10 | 73.43 | 18.23 | 24.65 | −224.39 | 140.90 | 1.11 | 21.70 |
| IL-6 (pg/dL) | −0.69 | 0.64 | 0.21 | 2.27 | −1.07 | 1.64 | 2.36 | 7.97 |
| PARP (pg/dL) | −13.28 | 15.33 | −5.02 | 17.23 | −10.63 | 18.26 | −2.97 | 8.87 |
| NF-κB (pg/dL) | −0.20 | 0.51 | −0.51 | 2.09 | −0.35 | 0.54 | −0.70 | 3.42 |
| Fox-O (pg/dL) | −1.53 | 1.99 | 2.48 | 7.44 | −0.50 | 2.06 | −0.65 | 5.45 |
| Insulin (U/dL) | −1.20 | 3.62 | 2.22 | 2.26 | −1.57 | 2.78 | 0.86 | 4.94 |
| MyoD (pg/dL) | −0.55 | 6.76 | −3.95 | 12.68 | 3.16 | 3.54 | −3.47 | 8.89 |
| PI3K (pg/dL) | 1.05 | 1.03 | 0.63 | 4.83 | 1.29 | 0.84 | 3.33 | 5.98 |
| mTOR (pg/dL) | 8.68 | 5.52 | 2.48 | 5.47 | 8.56 | 3.83 | 0.61 | 1.99 |
| IGF-1 (pg/dL) | 0.59 | 0.94 | −0.47 | 5.41 | 2.02 | 3.84 | −4.59 | 12.21 |
| pAkt (pg/dL) | 228.41 | 314.44 | 407.88 | 349.93 | 362.17 | 313.99 | 182.69 | 352.43 |

TABLE 19
Analysis, by age, of the differences of the subjects' muscle protein synthesis/degradation
regulating factors and cell differentiation and maturation factor after the test

| | 20-40 years of age | | | | 40-60 years of age | | | |
|---|---|---|---|---|---|---|---|---|
| | NB23 group | | Placebo group | | NB23 group | | Placebo group | |
| | Mean | SD | Mean | SD | Mean | SD | Mean | SD |
| TNFα (pg/dL) | −245.04 | 128.37 | 3.92 | 23.70 | −143.87 | 116.68 | 7.39 | 22.54 |
| IL-6 (pg/dL) | −1.22 | 1.48 | 2.55 | 8.01 | −0.69 | 1.33 | −0.06 | 0.18 |
| PARP (pg/dL) | −16.95 | 13.23 | −6.53 | 12.02 | −7.15 | 18.89 | 0.78 | 4.63 |
| NF-κB (pg/dL) | −0.34 | 0.59 | −1.03 | 3.54 | −0.26 | 0.46 | 0.33 | 0.66 |
| Fox-O (pg/dL) | −1.12 | 2.83 | 0.85 | 6.06 | −0.51 | 0.79 | −2.02 | 5.75 |
| Insulin (U/dL) | −1.43 | 3.18 | 1.39 | 4.90 | −1.48 | 2.93 | 0.85 | 2.71 |
| MyoD (pg/dL) | 3.13 | 3.82 | −5.25 | 9.14 | 0.90 | 5.78 | 0.64 | 10.90 |
| PI3K (pg/dL) | 1.31 | 0.82 | 2.57 | 5.74 | 1.12 | 0.98 | −0.29 | 0.51 |
| mTOR (pg/dL) | 8.95 | 4.25 | 1.00 | 3.64 | 8.24 | 4.50 | 1.49 | 2.53 |
| IGF-1 (pg/dL) | 2.69 | 4.26 | −6.06 | 10.51 | 0.47 | 1.23 | 4.70 | 9.71 |
| pAkt (pg/dL) | 376.36 | 340.79 | 230.44 | 360.19 | 265.66 | 287.79 | 285.08 | 383.24 |

TABLE 20
Analysis, by BMI, of the differences of the subjects' muscle protein synthesis/degradation
regulating factors and cell differentiation and maturation factor after the test

| | BMI < 24 kg/m² | | | | BMI ≥ 24 kg/m² | | | |
|---|---|---|---|---|---|---|---|---|
| | NB23 group | | Placebo group | | NB23 group | | Placebo group | |
| | Mean | SD | Mean | SD | Mean | SD | Mean | SD |
| TNFα (pg/dL) | −187.85 | 117.60 | 3.80 | 23.06 | −200.11 | 145.33 | 5.95 | 23.89 |
| IL-6 (pg/dL) | −0.83 | 1.82 | 2.52 | 9.45 | −1.07 | 0.98 | 1.27 | 3.45 |
| PARP (pg/dL) | −10.96 | 18.33 | −3.44 | 7.09 | −12.05 | 16.58 | −3.64 | 14.68 |
| NF-κB (pg/dL) | −0.24 | 0.41 | −0.21 | 1.03 | −0.36 | 0.61 | −1.06 | 4.17 |
| Fox-O (pg/dL) | 0.19 | 1.78 | −1.14 | 3.81 | −1.68 | 1.93 | 1.41 | 7.60 |
| Insulin (U/dL) | −0.08 | 2.45 | 0.38 | 2.83 | −2.63 | 2.99 | 2.03 | 5.40 |
| MyoD (pg/dL) | 2.42 | 3.24 | −7.06 | 6.03 | 1.67 | 6.14 | −0.42 | 11.67 |
| PI3K (pg/dL) | 1.11 | 0.81 | 1.72 | 3.28 | 1.30 | 0.98 | 3.54 | 8.75 |
| mTOR (pg/dL) | 9.29 | 4.18 | 0.19 | 1.34 | 8.00 | 4.47 | 2.01 | 4.32 |
| IGF-1 (pg/dL) | 2.58 | 4.39 | −6.98 | 11.10 | 0.72 | 1.62 | −0.37 | 10.58 |
| pAkt (pg/dL) | 358.63 | 351.01 | 329.04 | 316.52 | 288.77 | 288.28 | 168.85 | 391.54 |

It can be known from the results in Table 16 that after taking the Lactobacillus paracasei NB23 strain for 12 weeks, the subjects in the NB23 group showed significantly higher expression of MyoD and significantly lower expression of the muscle protein degradation regulating factors TNFα, NF-κB, and Fox-O than their counterparts in the placebo group; and that 12 weeks into the test, the subjects in the NB23 group showed a significant increase in expression of the myogenic regulatory factor mTOR whereas the subjects in the placebo group showed no significant difference in mTOR expression.

It can be known from the results in Table 17 that after taking the Lactobacillus paracasei NB23 strain for 12 weeks, the subjects in the NB23 group had a significantly lower serum insulin value than their counterparts in the placebo group. Moreover, the subjects in the NB23 group showed a significant decrease in TNFα and IL-6 while their counterparts in the placebo group showed an increase, and the subjects in the NB23 group showed a significant increase in expression of mTOR and MyOD while their counterparts in the placebo group showed either a decrease or a relatively small increase.

It can be known from the results in Table 18 that after taking the Lactobacillus paracasei NB23 strain for 12 weeks, both male and female subjects in the NB23 group showed a decrease in serum TNFα whereas their counterparts in the placebo group showed an increase. Also, both male and female subjects in the NB23 group showed a greater increase in serum mTOR level than their counterparts in the placebo group. Moreover, the female subjects in the NB23 group showed a significant increase in serum MyoD while their counterparts in the placebo group showed a decrease. In addition, the female subjects in the NB23 group showed a significantly greater decrease in serum TNFα than their male counterparts in the same group.

It can be known from the results in Table 19 that after taking the Lactobacillus paracasei NB23 strain for 12 weeks, the subjects aged 20-40 in the NB23 group showed a significant decrease in serum TNFα and insulin whereas their counterparts in the placebo group showed an increase in both. Moreover, the subjects aged 20-40 in the NB23 group showed an increase in serum MyoD, IGF-1, and mTOR while their counterparts in the placebo group showed either a decrease or a relatively small increase in each of the aforesaid factors. In addition, the subjects aged 40-60 in the NB23 group showed a significant decrease in serum TNFα while their counterparts in the placebo group showed an increase, and the subjects aged 40-60 in the NB23 group showed an increase in serum PI3K and mTOR while their counterparts in the placebo group showed either a decrease or a relatively small increase in each of the aforesaid factors. A comparison of the test results of the subjects in the different age groups of the NB23 group further reveals that after taking the Lactobacillus paracasei NB23 strain for 12 weeks, the subjects aged 20-40 showed a significantly greater decrease in serum TNFα than the subjects aged 40-60. It can be inferred from the foregoing results that regardless of the subjects' ages, administering the disclosed Lactobacillus paracasei NB23 strain for 12 weeks can lower the level of TNFα (which is an inflammation factor) in serum and increase the level of mTOR (which is a muscle protein synthesis regulating factor) in serum.

It can be known from the results in Table 20 that the subjects with BMI<24 kg/m2 in the NB23 group showed a significant increase in serum MyoD, IFG-1, and mTOR while their counterparts in the placebo group showed either a decrease or a relatively small increase in each of the aforesaid factors. Moreover, the subjects with BMI<24 kg/m2 in the NB23 group showed a significant decrease in serum TNFα while their counterparts in the placebo group showed an increase. The subjects with BMI≥24 kg/m2 in the NB23 group showed a significant decrease in serum TNFα and insulin while their counterparts in the placebo group showed an increase in both, and the subjects with BMI≥24 kg/m2 in the NB23 group showed a significantly greater increase in serum mTOR than their counterparts in the placebo group. In addition, after taking the Lactobacillus paracasei NB23 strain for 12 weeks, the subjects with BMI≥24 kg/m2 in the NB23 group showed a significant decrease in FOX-O while the subjects with BMI<24 kg/m2 in the same group showed an increase.

It can be inferred from the foregoing results that the Lactobacillus paracasei NB23 strain disclosed herein can lower the level of TNFα (which is an inflammation factor) in the subjects' serum and increase the activity of mTOR (which is a muscle protein synthesis factor) in the serum. That is to say, administering an effective amount of the Lactobacillus paracasei NB23 strain disclosed herein or a metabolite thereof to an individual can effectively improve the individual's inflammatory response or related diseases and thereby enhance the activity of the individual's muscle synthesis at the same time, producing the effect of treating or improving diseases caused by insufficient muscle mass.

What is claimed is:

1. A Lactobacillus paracasei NB23 strain, deposited at the Food Industry Research and Development Institute in Hsinchu City, Taiwan, on Oct. 4, 2023, and given the accession number BCRC911201, and deposited at Deutsche Sammlung von Mikroorganismen und Zellkulturen GmbH (DSMZ) under deposit number DSM 35133 on Sep. 5, 2024.

2. A composition, comprising an effective amount of the Lactobacillus paracasei NB23 strain of claim 1 or a metabolite thereof.

3. A method for treating or preventing sarcopenia, metabolic syndrome, and/or a cardiovascular disease, comprising: administering an effective amount of the Lactobacillus paracasei NB23 strain of claim 1, a metabolite thereof, or a composition containing the Lactobacillus paracasei NB23 strain or the metabolite to an individual in order to prevent the individual from having muscle loss or insufficient muscle mass or improve the individual's muscle loss or insufficient muscle mass.

4. The method for treating or preventing sarcopenia, metabolic syndrome, and/or a cardiovascular disease as claimed in claim 3, wherein the Lactobacillus paracasei NB23 strain, the metabolite, or the composition is used to inhibit expression of the individual's nuclear factor kappa-light-chain-enhancer of activated B cells (NF-κB) and forkhead box protein-O (Fox-O).

5. The method for treating or preventing sarcopenia, metabolic syndrome, and/or a cardiovascular disease as claimed in claim 3, wherein the Lactobacillus paracasei NB23 strain, the metabolite, or the composition is used to increase the individual's muscle mass.

6. The method for treating or preventing sarcopenia, metabolic syndrome, and/or a cardiovascular disease as claimed in claim 3, wherein the Lactobacillus paracasei NB23 strain, the metabolite, or the composition is used to activate a signaling pathway of the individual's insulin-like growth factor 1 (IGF-1).

7. The method for treating or preventing sarcopenia, metabolic syndrome, and/or a cardiovascular disease as claimed in claim 3, wherein the metabolic syndrome is associated with imbalance of at least one of the following risk factors: blood pressure, blood glucose, low-density lipoprotein cholesterol, and triglycerides.

8. The method for treating or preventing sarcopenia, metabolic syndrome, and/or a cardiovascular disease as claimed in claim 3, wherein the cardiovascular disease is caused by vascular sclerosis.
